(19) United States
(12) Patent Application Publication
(10) Pub. No.: US 2009/0076566 A1
(43) Pub. Date: Mar. 19, 2009

(54) Epidural Cortical Stimulation System Using Shape Memory Alloy

(76) Inventors: Thomas P. Osypka, Palm Harbor, FL (US); Hyoung-Ihl Kim, Jeonbug (KR); Yong-Il Shin, Iksan City (KR); Hyo-Joon Kim, Jeonju (KR)

Correspondence Address: EDWARDS ANGELL PALMER & DODGE LLP, P.O. BOX 55874, BOSTON, MA, 02205, US

(21) Appl. No.: 12/172,211

(22) Filed: Jul. 11, 2008

Related U.S. Application Data
(60) Provisional application No. 60/955,858, filed on Aug. 14, 2007.

(30) Foreign Application Priority Data
Jul. 11, 2007 (KR) 10-2007-0069539

(51) Int. Cl. A61N 1/36; A61N 1/05

(52) U.S. Cl. 607/45

(57) ABSTRACT

An epidural cortical stimulation system includes a stimulation body and a connecting lead. The stimulation body has a core formed of a shape-memory material, insulation provided around the core, defining an outer surface of the stimulation body, and at least one electrode arranged on the outer surface of the stimulation body, adapted and configured to contact the dura of a patient. The connecting lead extends from a proximal end of the body, and is adapted and configured for electrical communication with a control unit for providing power to the body electrical cortical stimulation.

### CROSS-REFERENCE TO RELATED APPLICATIONS

This application claims benefit of priority to U.S. Provisional Application No. 60/955,858, filed Aug. 14, 2007, and to Korean patent application No. 10-2007-0069539, filed Jul. 11, 2007. Each of the foregoing applications is incorporated herein by reference in its entirety.

### FIELD OF THE INVENTION

The present invention relates to cortical stimulation. More specifically, the present invention is directed to cortical stimulators made from shape-memory materials.

### BACKGROUND

Cortical stimulation of the brain is becoming an important tool to reactivate or to enhance the plasticity of the brain, in order to augment neurological recovery following brain injury.

A variety of brain electrodes are known in the art for applying to the brain cortex epidurally or intradurally. Of such devices, many are either too small to treat large regions of the brain, or require the relatively invasive step of opening a large portion of a patient's cranium, and accordingly may also require application of general anesthesia.

Coactivation of multiple regions of the brain is used to improve recovery following brain injury, such as following a stroke. Typically, to accomplish this one or more electrodes or electrode arrays covering a large area must be used. However, such multiple electrodes or electrode arrays may require multiple and/or large openings to be made in the cranium. Such openings may be considered relatively invasive by disturbing a relatively large portion of the cranium.

Thus, there remains a continued need in the art for a minimally invasive device capable of stimulating relatively large areas of a patient's brain. The present invention provides such a device and related methods, and is a solution to the aforementioned problems.

### SUMMARY OF THE INVENTION

The purpose and advantages of the present invention will be set forth in and apparent from the description that follows.

The invention includes, in one aspect, an epidural cortical stimulation system includes a stimulation body and a connecting lead. The stimulation body has a core formed of a shape-memory material, insulation provided around the core, defining an outer surface of the stimulation body, and at least one electrode arranged on the outer surface of the stimulation body, adapted and configured to contact the dura of a patient. The connecting lead extends from a proximal end of the body, and is adapted and configured for electrical communication with a control unit for providing power to the body electrical cortical stimulation.

Optionally, the core can be formed of a nickel-titanium alloy. The insulation provided around the core can be silicone. The at least one electrode can be arranged on the stimulation body so as to be flush with the outer surface defined by the insulation.

The core can be capable of transitioning from a first morphology to a second morphology upon being exposed to a predetermined temperature range. The predetermined temperature range can be about 37 degrees Centigrade. The first morphology can occur at a temperature that allows the core to remain in a martensite phase. The second morphology can occur at a temperature that allows the core to transition to an austenite phase.

The at least one electrode can be electrically connected to the core by an intermediate conductive element.

The core can be electrically conductive and can be in electrical communication with the connecting lead and the at least one electrode. If desired, a conductor, separate from the core, can be provided in the body and is in electrical communication with the connecting lead and the at least one electrode.

The connecting lead can be adapted and configured to extend through an aperture formed in the cranium of a patient.

In accordance with a further aspect of the invention, a method for cortical stimulation includes the steps of providing a cortical stimulation system having a body arranged in at a first morphology, forming an aperture in a patient's cranium, inserting the body through the aperture, between the cranium and dura of the patient, and allowing the body to transition from the first morphology to a second morphology due to the effect of body temperature.

It is to be understood that both the foregoing general description and the following detailed description are exemplary and are intended to provide further explanation of the invention.

### BRIEF DESCRIPTION OF THE DRAWINGS

The accompanying drawings, which are incorporated in and constitute part of this specification, are included to illustrate and provide a further understanding of the invention. Together with the description, the drawings serve to explain the principles of the invention, wherein:

FIG. 1 is a top view of an epidural cortical stimulator in accordance with the present invention, where the stimulator is illustrated in a first morphology;

FIG. 2 is a top view of the epidural cortical stimulator of FIG. 1, where the stimulator is illustrated in a second morphology;

FIG. 3 is a bottom view of the epidural cortical stimulator of FIG. 1, where the stimulator is illustrated in the first morphology, and illustrates the electrodes carried by the epidural cortical stimulator;

FIG. 4 is a cross-sectional view of the epidural cortical stimulator of FIG. 1, taken across a point on the epidural cortical stimulator that does not include an electrode;

FIG. 5 is a cross-sectional view of the epidural cortical stimulator of FIG. 1, taken across a point on the epidural cortical stimulator that does include an electrode;

FIGS. 6 and 7 are bottom views of the epidural cortical stimulator of FIG. 1, illustrating a change in shape of the epidural cortical stimulator at a predetermined temperature;

FIG. 8 is a partial cross-sectional view of a patient's cranium and brain cortex, illustrating placement of the epidural cortical stimulator of FIG. 1 between the dura and the cranium of the patient, while in a first morphology;

FIG. 9 is a partial cross-sectional view of a patient's cranium and brain cortex, illustrating the epidural cortical stimulator of FIG. 1 in a second morphology, arranged between the dura and the cranium of the patient;

FIG. 10 is a top view of a patient's brain, illustrating placement of the epidural cortical stimulator of FIG. 1 thereon in the second morphology, and relative arrangement of electrodes with respect to the brain cortex;

FIG. 11 illustrates placement of the epidural cortical stimulator of FIG. 1 thereon in the second morphology in an alternate area of the patient's brain; and

FIG. 12 illustrates placement of the epidural cortical stimulator of FIG. 1 thereon in the second morphology in still another alternate area of the patient's brain.

### DETAILED DESCRIPTION OF THE PREFERRED EMBODIMENT

Reference will now be made in detail to example embodiments of the invention, which are illustrated in the accompanying drawings. The methods of the invention will be described in conjunction with the related devices.

The devices and methods presented herein may be used for cortical stimulation of the brain for any of a number of brain disorders, including epilepsy or to enhance recover following brain injury, for example. The present invention provides a minimally invasive device capable of stimulating a relatively large area of the brain cortex without necessitating forming a large opening in the patient's cranium. Further, epidural cortical stimulators in accordance with the invention are easily removed from the patient, without necessitating re-opening a large portion of the patient's cranium.

The epidural cortical stimulator 100, as seen in FIGS. 1-12, includes a stimulation body 110, and a connecting lead 120 joined to the body 110 by an optional intermediate connection 140. The connecting lead 120 can be of any necessary length to reach a power control unit, and terminates at its distal end in a connecting terminal 130.

The core 113 is made from a shape-memory material, such as shape-memory metallic alloys, and may be made from a nickel-titanium alloy, for example. However, other shape-memory materials can be used. The core 113 is preferably made from an electrically conductive shape-memory material, which advantageously allows the core 113 to carry current to one or more electrodes 117. If the core 113 is formed of a non-conductive material, then a separate conductor can be provided in the stimulator body 110 to deliver current to the electrodes 117.

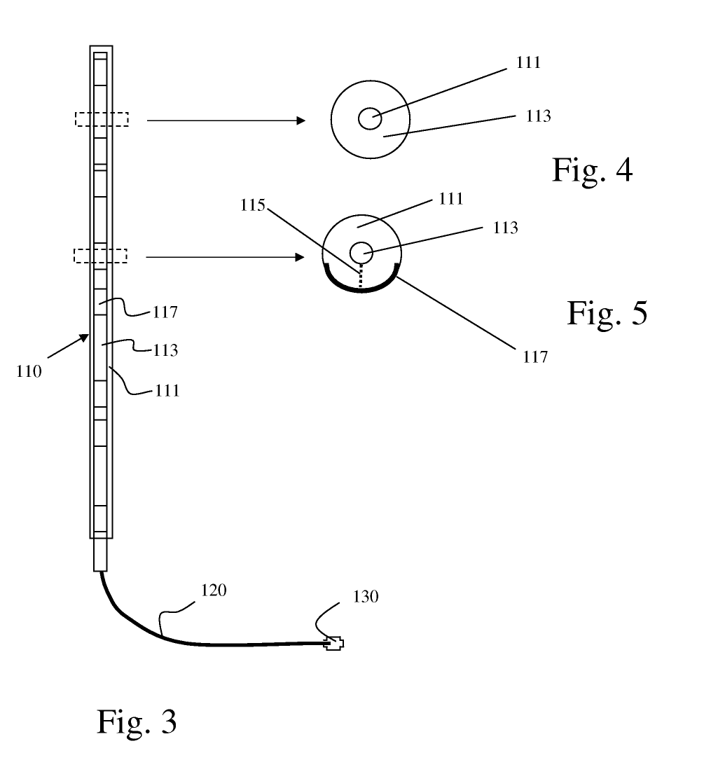

As best seen in FIGS. 3-5, insulation 111 is provided on and surrounds the core 113. The insulation can be any suitable flexible biocompatible electrically insulating material, such as silicone. One or more electrodes 117 are provided on the outer surface of the stimulator 100 for contacting the dura of the patient's brain. The electrodes 117 can be formed from any suitable biocompatible and electrically conductive material. Example materials for this use are shape-memory metallic alloys such as nickel-titanium alloys, other nickel or titanium alloys, stainless steel or the like. Although it is envisioned that the stimulator 100 will typically be used epidurally, it should be understood that there may be situations where the device is appropriate for use intradurally.

The electrodes 117 can also be integrally formed with an internal structure, such as an internal spine or cannula, which may include an open framework as a structure.

If the core 113 is made from an electrically-conductive material, the electrodes 117 are in electrical communication with the core 113 by way of intermediate conductor 115 or other suitable electrical connection. In alternate embodiments, the electrode 117 can have an integral protrusion that extends from the electrode and contacts the core.

The core 111 can be formed of the shape-memory material, after which the core 111 can be insert molded into the insulation 113 or made by any other suitable method. Likewise, the electrodes 117 can be insert molded into the insulation 111 during the same step.

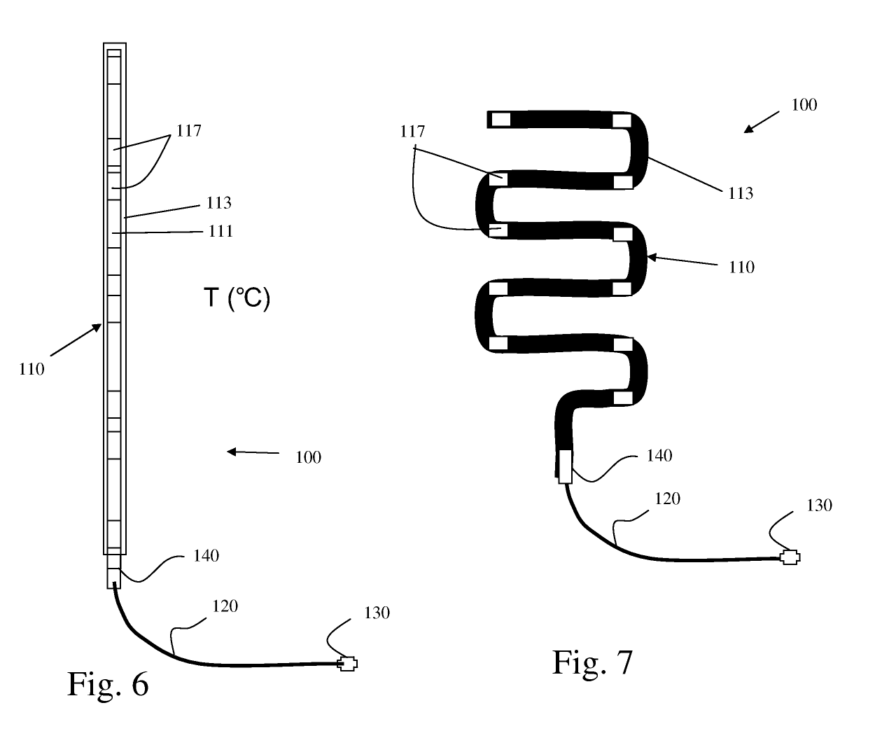

FIGS. 6 and 7 are bottom views of the cortical stimulator 100 in accordance with the invention, which illustrate one example arrangement of electrodes 117 along the body 110, where when the body 110 is in its second morphology, as illustrated in FIG. 7, the electrodes 117 are arranged in a rectangular array. The length of the body 110 and spacing between electrodes 117 can be selected so as to provide an array of any practical size, with electrodes in the pattern desired. Naturally, other configurations can be provided in accordance with the invention, such as a circular array of electrodes, with the body 110 being provided in a spiral configuration in its second morphology. The invention is not limited to any one particular morphology of the body 110. As mentioned hereinabove, multiple stimulators 100 can be used in order to coactivate different areas of the brain.

Removal of the stimulator 100 simply requires a gentle pulling force applied to the distal end or to the connecting lead 120. Such removal is much less invasive than methods for removing electrodes of the prior art.

Shape memory effects can be imparted on the stimulator 100 either before or after forming the insulating layer 113 on the core 111. In the case of nickel-titanium alloys, which exhibit their shape-memory properties due to a phase change in their crystal structure, the desired ultimate shape can be imparted while the material is in its austenite phase. The material can then be cooled to its martensite phase, and deformed to a first morphology, which can simply be a shape to facilitate packaging, or can be a shape to facilitate insertion through the cranium of the patient. The material used for the core 111 is preferably selected such that when it warms to body temperature (about 37 degrees Centigrade) it reverts back to the austenite phase and the shape previously imparted thereon while in the austenite phase.

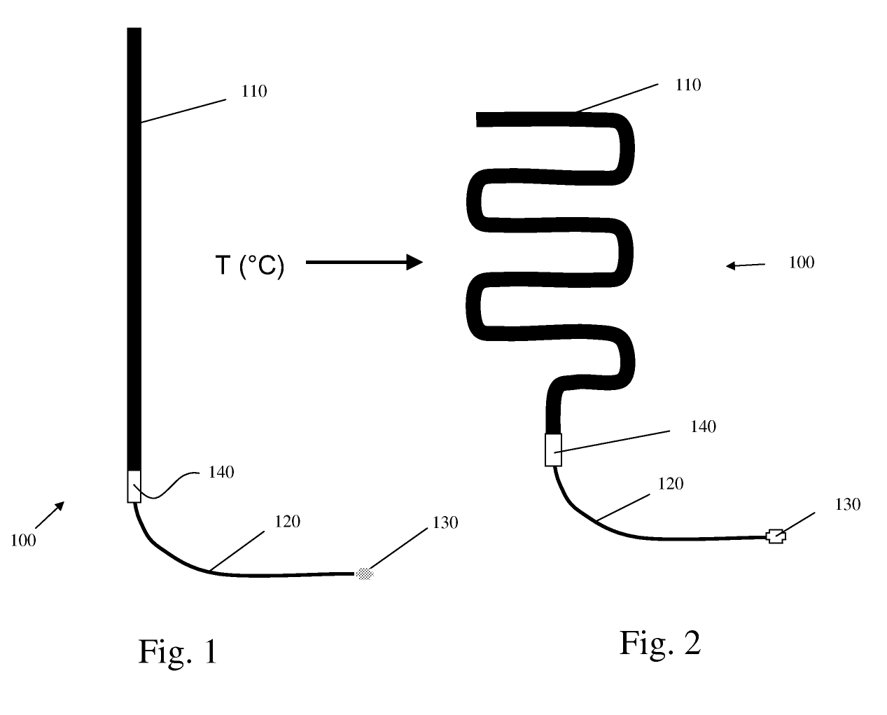

The above-described transition in shape is illustrated in FIGS. 1 and 2, and FIGS. 6 and 7, each pair of figures illustrating top or bottom views respectively. In FIGS. 1 and 6, the stimulator body 110 is provided in a first morphology, in this case, straight. The cortical stimulator 100 can be packaged with the body 110 in this configuration, or the physician can manipulate the body 110 into this or any shape necessary to facilitate insertion. In any case, the body 110 will revert to its intended final or “second” morphology, an example of which is illustrated in FIGS. 2 and 7, illustrating an overall serpentine shape.

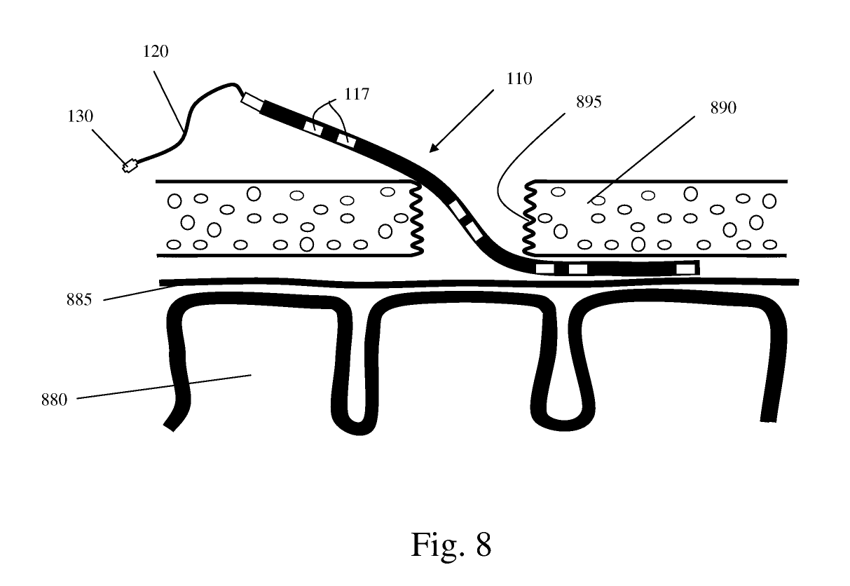

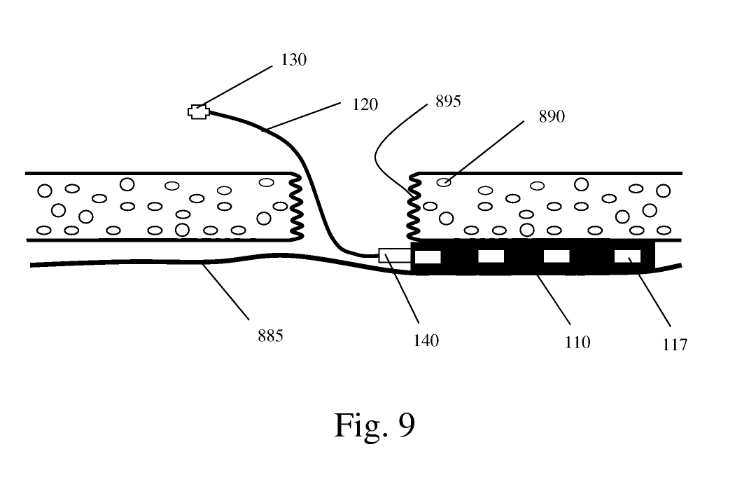

FIGS. 8 and 9 illustrate the process of inserting the cortical stimulator 100 between the cranium 890 and the dura 885 of the brain 880 of a patient. An aperture 895 is formed in the cranium 890 by way of a suitable surgical method, and need not be very large. A small burr hole may be sufficient for insertion. The body 110 is then advanced through the aperture 895, between the dura 885 and the cranium 890. The body temperature of the patient then causes the body 110 of the cortical stimulator 100 to revert to its second morphology, due the shape-memory effect of the core 113, as illustrated in FIG. 9.

It is envisioned, however that materials other than shape-memory materials can be used to effect a change in morphology of the body 110. Such materials, for example can include materials that exhibit a deformation due to application of electrical current, where a separate core of such material, not in electrical contact with the electrodes 117, is provided in a separate electrical circuit to which a current is applied to effect a shape change to the desired second morphology. A piezoelectric material can be used for this purpose, for example.

If desired, distinctive markings can be provided on the stimulator body 110 so that the upper surface of the cortical stimulator 100 and lower surface having electrodes 117 can be easily distinguished.

In alternate embodiments, the intermediate connection 140 may simply be a continuation of the core 113, or may be welded to the core 113, in order to form a seamless component, with a low-profile continuous insulating jacket.

Further, the connecting terminal 130 may be connected to a pulse generator (not shown) arranged in a convenient location, such as a low-profile generator placed in the cranium, or a pace-maker style control unit placed in the chest wall of the patient. Alternatively still, the connecting terminal 130 can be connected to an induction coil that receives power through induction from an external device.

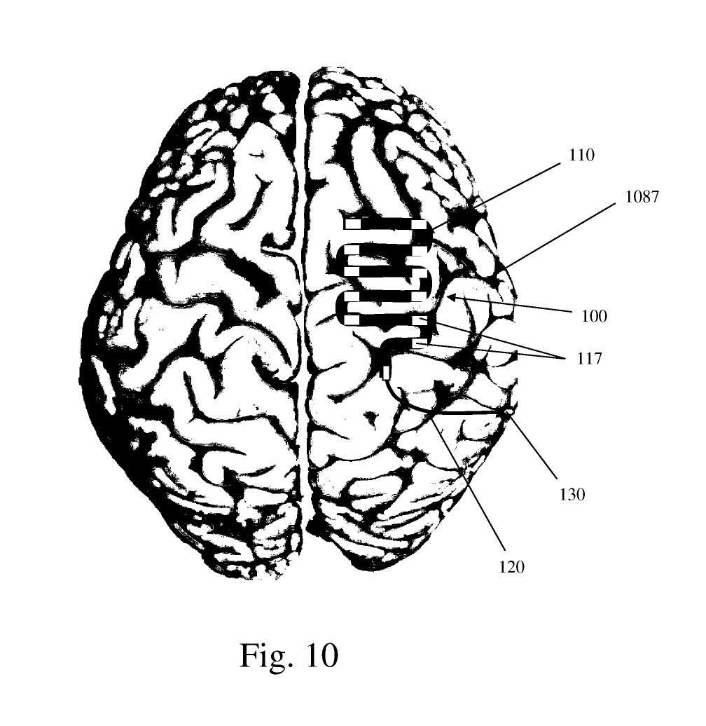

FIGS. 10-12 illustrate the cortical stimulator 100 in accordance with the invention arranged in a second morphology, and arranged in various locations with respect to a brain 1087.

FIG. 10 illustrates the cortical stimulator 100 arranged on the upper region of the brain 1087 in the premotor and motor cortex areas for treatment of chronic central pain or chronic stroke.

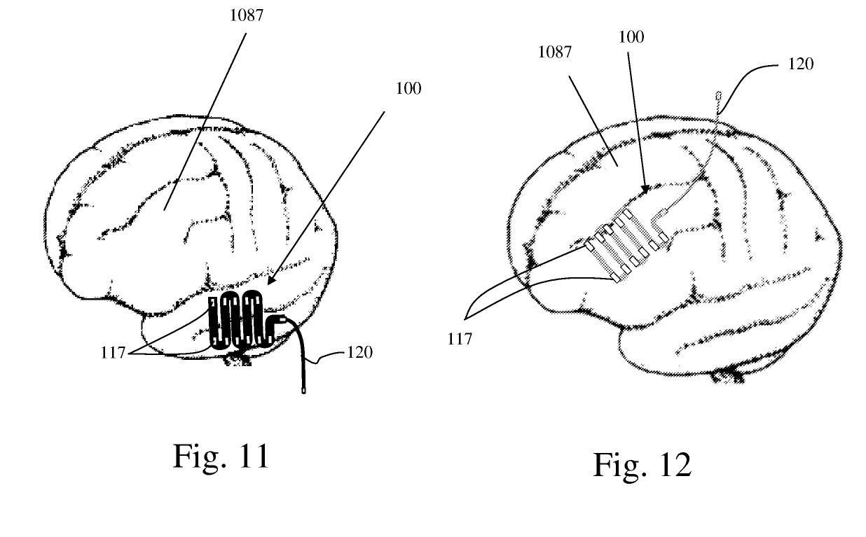

FIG. 11 illustrates the cortical stimulator 100 arranged on the temporal lobe region of the brain 1087 for treatment of temporal lobe epilepsy.

FIG. 12 illustrates the cortical stimulator 100 arranged on the upper region of the brain 1087 in the precentral area for treatment of depression or Alzheimer's disease.

The devices and methods of the present invention, as described above and shown in the drawings, provide for a cortical stimulators with superior properties. It will be apparent to those skilled in the art that various modifications and variations can be made in the device and method of the present invention without departing from the spirit or scope of the invention. Thus, it is intended that the present invention include such modifications and variations.

What is claimed is:

1. An epidural cortical stimulation system comprising:
a) a stimulation body having:
i) a core formed of a shape-memory material;
ii) insulation provided around the core, defining an outer surface of the stimulation body; and
iii) at least one electrode arranged on the outer surface of the stimulation body, adapted and configured to contact the dura of a patient; and
b) a connecting lead extending from a proximal end of the body, adapted and configured for electrical communication with a control unit for providing power to the body electrical cortical stimulation.

2. The epidural cortical stimulation system of claim 1, wherein the core is formed of a nickel-titanium alloy.

3. The epidural cortical stimulation system of claim 1, wherein the insulation provided around the core is silicone.

4. The epidural cortical stimulation system of claim 1, wherein the at least one electrode is arranged on the stimulation body so as to be flush with the outer surface, defined by the insulation.

5. The epidural cortical stimulation system of claim 1, wherein the core is capable of transitioning from a first morphology to a second morphology upon being exposed to a predetermined temperature range.

6. The epidural cortical stimulation system of claim 5, wherein the predetermined temperature range is about 37 degrees Centigrade.

7. The epidural cortical stimulation system of claim 5, wherein the first morphology occurs at a temperature that allows the core to remain in a martensite phase.

8. The epidural cortical stimulation system of claim 5, wherein the second morphology occurs at a temperature that allows the core to transition to an austenite phase.

9. The epidural cortical stimulation system of claim 1, wherein the at least one electrode is electrically connected to the core by an intermediate conductive element.

10. The epidural cortical stimulation system of claim 1, wherein the core is electrically conductive and is in electrical communication with the connecting lead and the at least one electrode

11. The epidural cortical stimulation system of claim 1, wherein a conductor, separate from the core, is provided in the body and is in electrical communication with the connecting lead and the at least one electrode.

12. The epidural cortical stimulation system of claim 1, wherein the connecting lead is adapted and configured to extend through an aperture formed in the cranium of a patient.

13. A method for cortical stimulation, the method comprising the steps of:
a) providing a cortical stimulation system having a body arranged in at a first morphology;
b) forming an aperture in a patient's cranium;
c) inserting the body through the aperture, between the cranium and dura of the patient; and
d) allowing the body to transition from the first morphology to a second morphology due to the effect of body temperature.
